(19) United States
(12) Patent Application Publication
(10) Pub. No.: US 2005/0002863 A1
(43) Pub. Date: Jan. 6, 2005

(54) METHOD FOR ASSESSING FOOD PALATABILITY AND PREFERENCE IN ANIMALS USING A COGNITIVE PALATABILITY ASSESSMENT PROTOCOL

(75) Inventors: Joseph Araujo, Toronto (CA); Bill Millgram, 1265 Military Trail (CA)

Correspondence Address: DALINA LAW GROUP, P.C., 7910 IVANHOE AVE. #325, LA JOLLA, CA, 92037, US

(73) Assignee: CANCOG TECHNOLOGIES INC., Scarborough (CA)

(21) Appl. No.: 10/710,337

(22) Filed: Jul. 1, 2004

Related U.S. Application Data
(60) Provisional application No. 60/483,639, filed on Jul. 1, 2003.

(51) Int. Cl. A61K 49/00

(52) U.S. Cl. 424/9.2

(57) ABSTRACT

Embodiments of the invention relate to a novel approach for determining the palatability, or preference, of foods, food stuffs, or veterinary biologics in animals. This approach provides a robust and reliable means of assessing palatability in animals using a cognitive palatability assessment protocol. The basic protocol utilizes a discrimination learning procedure, in which animals are presented with three simultaneous stimuli, such as objects, odors, sounds, digital pictures or projections, etc., and are allowed to respond to one, although any number of stimuli may be employed. The response is then recorded including metadata such as the rate of learning, response rate and response order for example. Depending on the animal's choice, the response will typically result in the animal receiving no reward, or either of two particular foods, food stuffs or veterinary biologics. The animal's response selection is indicative of a preference for one food, food stuff or veterinary biologic over the other and is indicative of the palatability of one food, food stuff or veterinary biologic in comparison to the other. This method requires the use of fewer animals than alternative approaches and may prove healthier for animals that do not self-limit their food consumption.

### BACKGROUND OF INVENTION

This application claims priority from U.S. Provisional Application 60/483,639 filed Jul. 1, 2004 entitled “ASSESSMENT OF FOOD PALATABILITY WITH A DISCRIMINATION LEARNING PROCEDURE” which is hereby incorporated herein by reference.

### FIELD OF THE INVENTION

Embodiments of the invention described herein relate generally to a method for assessing food palatability and preference in animals using a cognitive palatability assessment protocol. More particularly, but not by way of limitation, these embodiments yield improved pet food compositions and provide methods for determining the palatability and preference of such compositions to animals.

### DESCRIPTION OF THE RELATED ART

Palatability of a food, food stuff, or veterinary biologic is a measure of subjective hedonic preference and depends on both taste and odor. Assessment of palatability is important for both the veterinary and the pet-food industries. The greater the palatability of a food, food stuff or veterinary biologic, the easier the administration and the more enjoyable the experience. Because animals are unable to declare preferences and hedonic value verbally, the assessment of palatability must be based on an objective measure in which two or more foods can be ranked on the basis of preference. The most common method of assessing palatability in canines is with a two-choice preference test. Typically, this involves introducing two bowls of different foods to the animal and calculating the amount of each consumed over a specified period of time (see Ferrell 1984a; Ferrell 1984b; Griffin et al. 1984). The food ingested in higher quantities is considered to be more palatable and the preferred food. This procedure allows palatability to be determined rapidly, but with several confounding problems. First, the grouped data shows high variability, requiring large sample sizes to achieve statistical significance. Second, the individual animal data are unreliable, possibly because the animal simply continues eating the food they sampled first. Third, the procedure doesn't control for food interactions in which the presence of one food may alter the palatability of the other. Fourth, satiety effects are not controlled. For example, some foods may produce rapid satiety, or feelings of fullness, thereby decreasing the total amount consumed. The two-pan test is also unable to deal with the dynamic aspects of palatability, by which the palatability of any given food varies over time. This confound could potentially be dealt with using a long-term analysis of total food intake; however, this can confound differences in palatability with differences in caloric or nutritional values of meals (Sunday et al. 1983). In addition, the large amount of food consumed using the two-pan test may reduce its utility in long-term testing and may be unhealthy for animals over time, particularly those that do not self-limit their food intake.

A second two-choice approach for studying canine food preference employs a concurrent-schedule procedure, based on an operant-conditioning paradigm, to assess the strength of an animal's motivation to eat (see Chao 1984; Rashotte et al. 1984). A preference of one food over another is established by an animal's pressing one lever more frequently than the other (Rashotte & Smith 1984). Although this methodology circumvents many of the problems associated with the two-pan test, it is time consuming and the data are difficult to analyze. More problematic, the concurrent-schedule procedure is less robust than the two-pan test; preference scores obtained were weaker than those obtained in two-pan tests, even when the foods tested differed significantly in appearance and texture (Rashotte et al. 1984). While this procedure may allow hedonic-value scaling of foods, the time duration required, the difficulty in interpretation, and the highly variable results make it an impractical test to determine food palatability or preference.

Both the two-pan test and the concurrent schedule paradigm assume that a food preference is established when an animal responds to only one side, e.g., eating the food in the left pan or responding only to the left lever. Typically, animals attempt to obtain as much food as possible; a confound that is particularly important when the foods do not differ greatly in palatability. Thus, once a dog starts eating from the left pan, it may continue until it has consumed all of the available food.

### SUMMARY OF INVENTION

Embodiments of the invention relate to a novel approach for determining the palatability, or preference, of foods, food stuffs, or veterinary biologics in animals. This approach provides a robust and reliable means of assessing palatability in animals using a cognitive palatability assessment protocol. The basic protocol utilizes a discrimination learning procedure, in which animals are presented with three simultaneous stimuli, such as objects, odors, sounds, digital pictures or projections, etc., and are allowed to respond to one, although any number of stimuli may be employed. The response is then recorded including metadata such as the rate of learning, response rate and response order for example. Depending on the animal's choice, the response will typically result in the animal receiving no reward, or either of two particular foods, food stuffs or veterinary biologics. The animal's response selection is indicative of a preference for one food, food stuff or veterinary biologic over the other and is indicative of the palatability of one food, food stuff or veterinary biologic in comparison to the other. This method requires the use of fewer animals than alternative approaches and may prove healthier for animals that do not self-limit their food consumption.

The cognitive method described herein possesses a higher resolution of detecting differences in palatability than other palatability-assessment procedures. The cognitive method also controls for a variety of non-cognitive factors, including satiety and the possibility that the palatability of one food affects the preference for the other, by using a forced-choice paradigm and by limiting the amount of food consumed. Furthermore, this novel approach to palatability testing can be varied to examine short- or long-term preferences without nutritional, caloric or satiety-related confounding effects. Other factors that can also be studied using this cognitive method include pre- and post-feeding, temporal differences, and changes induced by hormonal or pharmacological treatment.

This approach provides an objective measure of food preference using a limited number of animals while controlling for other factors influencing feeding, such as satiety. Furthermore, the cognitive test protocol can be modified to examine the contribution of other factors to animal food preference, such as age, hormonal or pharmacological state, and dietary experience. Embodiments of the invention may optionally use a non-rewarded object. The absence of responses to the non-rewarded object further indicates learning, and provides an essential control for the possibility of an animal having no preference between the foods, food stuffs or veterinary biologics.

Embodiments of the invention also relate to the production of compositions, including foods, food stuffs, pharmaceutical compositions and veterinary biologics having enhanced palatability to an animal wherein the compositions are selected in accordance with the methods described herein.

The cognitive palatability assessment protocol comprises phases such as the optional preference phase, optional association phase, discrimination phase, optional stabilization phase and optional reversal phase.

The optional preference phase comprises presenting an animal with at least one distinct stimulus wherein each stimulus is associated with an identical reward. The animal chooses a stimulus, or stimuli, wherein choice of any one stimulus results in a reward. The preferred object is recorded and in some embodiments of the invention utilized with no reward if chosen by the animal.

The optional association phase comprises presenting an animal with at least one stimulus associated with a reward in order to enable the animal to associate a reward with a choice.

The discrimination learning phase comprises a discrimination learning procedure comprising presenting an animal with at least one stimulus and permitting the animal to choose a stimulus wherein the choice is recorded in order to further establish a preferred food and a non-preferred food. Some embodiments of the invention utilize three stimuli comprising one preferred stimuli and two non-preferred stimuli wherein choice of the preferred stimuli yields no reward and wherein choice of a non-preferred stimuli results in a reward.

The optional stabilization phase comprises repetition of the discrimination learning phase within sessions for a number of sessions.

The optional reversal phase comprises switching preferred and non-preferred food associations with respect to the associated stimuli.

In this document the following terminology is adopted: The term “discrimination learning” refers to learning manifested in the ability to respond differentially to various stimuli. Examples of various stimuli include, but are not limited to, objects that differ in shape, color or a combination of both, light differing in intensity, hue or frequency may also be used as a stimulus. Also odors, differences in texture, and sounds can be used as stimuli. Any stimulus that can be discriminated from another can be used as a stimulus.

The term “foodstuff” refers to a substance that can be used or prepared for use as food. Examples include, but are not limited to, dry and wet feed, flavor enhancers, and nutritional supplements.

The term “palatability” encompasses all of the various properties of foods, foodstuffs and veterinary biologics that are sensed by the consuming animal and determine the overall acceptability, or preference, of the food. These properties include, but are not limited to, texture, taste, and aroma.

The term “preference” refers to an animals preferred food, food stuff, or veterinary biologic when compared to another food, food stuff or veterinary biologic.

The term “veterinary biologic” means (a) a helminth, protozoa or micro-organism, (b) a substance or mixture of substances derived from animals, helminths, protozoa or micro-organisms, or (c) a substance of synthetic origin that is manufactured, sold or represented for use in restoring, correcting or modifying organic functions in animals or for use in the diagnosis, treatment, mitigation or prevention of a disease, disorder or abnormal physical state, or the symptoms thereof, in animals.

### BRIEF DESCRIPTION OF DRAWINGS

FIG. 1 shows an embodiment of reward configurations used in the object-reward test protocol for establishing dietary preferences. In this example, one object (e.g., the small block) is always associated with a moist food reward, and second object (e.g., large cylinder) is associated with dry food reward. The third object (e.g., large block) is associated with no food reward. The figure shows the 6 possible spatial configurations that are used, each twice per daily test session.

FIG. 2 shows training and stabilization data with responses to each of the three objects on each test session over the initial training and stabilization sessions for one subject. The arrow indicates the point at which the subject reaches criterion on the conditioning phase. The dog for which the data is displayed rapidly developed a preference for responding to the object associated with the moist test diet, and this preference was maintained over the stabilization session.

FIG. 3 shows performance of one subject on initial and reversal learning. During initial learning, the dog rapidly learned to approach the object associated with the moist diet as per Table 1. After switching the objects associated with the dry reward and moist diet, the dog modified its object preference.

FIG. 4 shows the sum of food choices over phase 1 and 2. The number of choices of the moist food was significantly higher than either the non-reward or the dry food (p<0.05 in both cases).

FIG. 5 shows an embodiment of a method of performing a preference phase.

FIG. 6 shows an embodiment of a method of performing an association phase.

FIG. 7 shows an embodiment of a method of performing discrimination conditioning.

FIG. 8 shows an embodiment of a method of performing a reversal phase.

### DETAILED DESCRIPTION

Embodiments of the invention provide a rapid, reproducible, and sensitive quantitative cognitive method for determining the palatability and preference of foods, foodstuffs and veterinary biologics, whether natural or synthetic, complex or simple, presented singly or in combination, to a wide variety of animals including canines. This approach provides a robust and reliable means of assessing palatability in animals using a cognitive palatability assessment protocol. The basic protocol utilizes a discrimination learning procedure, in which animals are presented with three simultaneous stimuli, such as objects, odors, sounds, digital pictures or projections, etc., and are allowed to respond to one, although any number of stimuli may be employed. The response is then recorded including metadata such as the rate of learning, response rate and response order for example. Depending on the animal's choice, the response will typically result in the animal receiving a no reward, or either of two particular foods, food stuffs or veterinary biologics. The animal's response selection is indicative of a preference for one food, food stuff or veterinary biologic over the other and is indicative of the palatability of one food, food stuff or veterinary biologic in comparison to the other.

Embodiments of the invention optionally comprise a single or multiple stimulus preference test(s). In one embodiment of the invention the stimuli are objects of different shape, color, size or a combination thereof. In another embodiment of the invention the stimuli are lights of different color, hue, intensity frequency or a combination thereof. In yet other embodiments, the stimuli may be distinct sounds, odors, or varying textures; any discernable stimuli may be used. The preference test is optional. As an example, response rates (latency to respond or frequency of responses) to a single stimulus associated with a single food, food stuff or veterinary biologic could also be used to assess food, food stuff or veterinary biologic preference, when compared to response rates (latency to respond or frequency of responses) to a second stimulus associated with another food, food stuff or veterinary biologic. Similarly, the discrimination procedure may be used in the absence of assessing stimulus preferences, but identifying preferred stimuli affords a more robust interpretation of the discrimination results. Similarly, two distinct stimuli, or more, associated with foods, food stuffs or veterinary biologics may be used, but the presence of a non-rewarded stimulus affords control of the animal having no preference, the indication of learning, or the absence of motivation. Ideally, three or more distinct stimuli should be used, in which the preferred stimulus is not rewarded in the discrimination learning procedure.

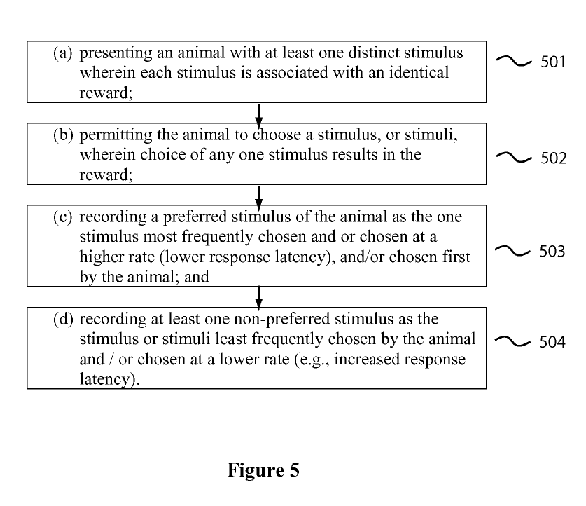

In one embodiment of the invention as shown in FIG. 5 the discrimination procedure further comprises a single or multiple preference test(s) comprising the steps of: (a) presenting an animal with at least one distinct stimulus wherein each stimulus is associated with an identical reward at 501; (b) permitting the animal to choose a stimulus, or stimuli, wherein choice of any one stimulus results in the reward at 502; (c) recording a preferred stimulus of the animal as the one stimulus most frequently chosen and or chosen at a higher rate (lower response latency), and/or chosen first by the animal at 503; and (d) recording at least one non-preferred stimulus as the stimulus or stimuli least frequently chosen by the animal and/or chosen at a lower rate (e.g., increased response latency) at 504.

In another embodiment of the invention the discrimination procedure further comprises replacement of step (d) of the preference test with recording at least two non-preferred stimuli as the stimuli least frequently chosen by the animal and/or chosen at a lower rate (increased response latencies).

In another embodiment of the invention the discrimination procedure further comprises replacement of step (a) of the preference test with presenting an animal with at least two distinct stimuli wherein each stimulus is associated with an identical reward; One embodiment of the invention may further comprise 5-1000 association sessions. Another embodiment of the invention may further comprise 3-4 association sessions. In another embodiment of the invention, the first phase comprises 1-2 association sessions, and in yet another embodiment of the invention, the first phase does not comprise any association sessions. The association tests may increase the rate of subsequent discrimination conditioning, but is not required for successful interpretation of the results.

In one embodiment of the invention, the discrimination procedure further comprises at least one association session comprising: (a) presenting the animal with at least one stimulus associated with the test food, food stuff or veterinary biologic; and (b) permitting the animal to choose the stimulus wherein choice of the stimulus results in reward of the particular test food, food stuff or veterinary biologic; (c) optionally repeating steps (a) and (b) until association is achieved with the same stimulus, or other stimuli, associated with the same or other foods, food stuffs or veterinary biologics.

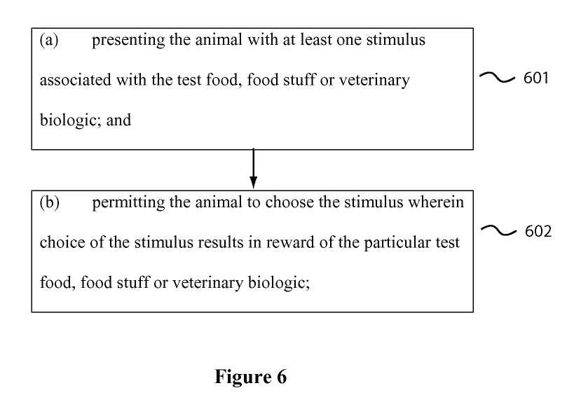

In another embodiment of the invention as shown in FIG. 6 the discrimination procedure further comprises at least one association session comprising: (a) presenting the animal with at least one non-preferred stimulus associated with the test food, food stuff or veterinary biologic at 601; (b) permitting the animal to choose the non-preferred stimulus wherein choice of the non-preferred stimulus results in reward of the particular test food, food stuff or veterinary biologic at 602; and, (c) optionally repeating steps (a) and (b) until association is achieved with the same stimulus, or other stimuli, associated with the same or other foods, food stuffs or veterinary biologics.

One embodiment of the invention uses a discrimination learning procedure wherein the animals are trained on a 10-20 choice stimuli discrimination learning procedure. In another embodiment of the invention the animals are trained on a 4-10 choice stimuli discrimination learning procedure. In another embodiment of the invention, the animals are trained on a single stimulus choice discrimination learning procedure in parallel, simultaneously or concurrently. In yet another embodiment the animals are trained on a 2-choice discrimination learning procedure.

In one embodiment of the invention, the discrimination learning procedure comprises: (a) presenting the animal with at least one distinct stimulus; (b) permitting the animal to choose the stimulus or stimuli wherein choice of a stimulus results in reward of a particular food, a food stuff, or veterinary biologic; (c) recording the stimulus chosen by the animal and/or the latency to respond, and/or the order of responses; (d) repeating steps (a) through (c) if the desired level of discrimination conditioning of the animal is not yet achieved; (e) establishing a preferred food, food stuff, or veterinary biologic of the animal as the food, food stuff, or veterinary biologic associated with the highest frequency and/or response rate and/or rate of learning, and/or combination thereof, of stimulus choice; and, (f) establishing the non-preferred food, foodstuff, or veterinary biologic as the food, food stuff, or veterinary biologic associated with lower frequency and/or response rate, and/or learning rate, and/or combination thereof, of stimulus choice.

Steps (a) through (d) may be repeated with individual stimuli either in parallel, concurrently or sequentially and the number of trials that are used to establish the preference may vary.

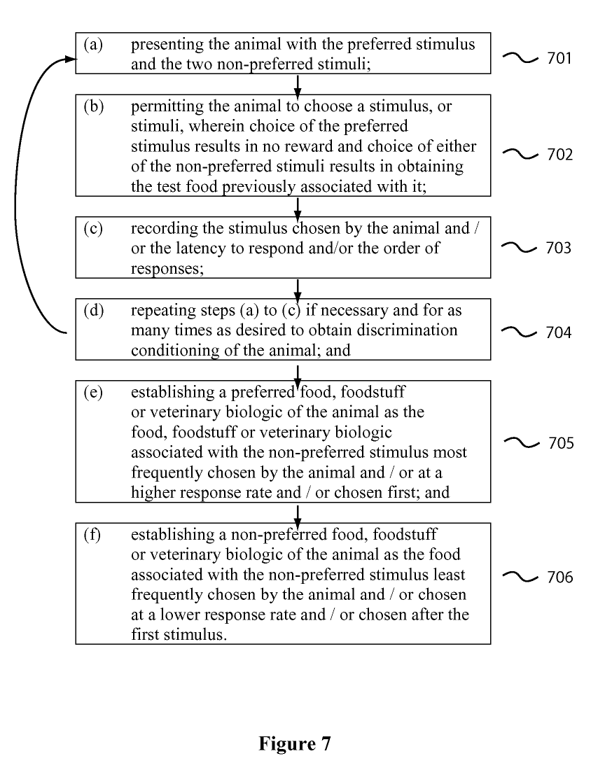

In another embodiment of the invention, the discrimination learning procedure comprises replacement of step (a) with presenting the animal with at least two distinct stimuli; In another embodiment of the invention as shown in FIG. 7 each animal is trained on a 3 choice stimuli discrimination learning procedure comprising: (a) presenting the animal with the preferred stimulus and the two non-preferred stimuli at 701; (b) permitting the animal to choose a stimulus, or stimuli, wherein choice of the preferred stimulus results in no reward and choice of either of the non-preferred stimuli results in obtaining the test food previously associated with it at 702; (c) recording the stimulus chosen by the animal and/or the latency to respond and/or the order of responses at 703; (d) repeating steps (a) to (c) if necessary and for as many times as desired to obtain discrimination conditioning of the animal at 704; (e) optionally, establishing a preferred food, foodstuff, or veterinary biologic of the animal as the food, foodstuff or veterinary biologic associated with the non-preferred stimulus most frequently chosen by the animal and/or at a higher response rate and/or chosen first at 705; and (f) optionally, establishing a non-preferred food, foodstuff or veterinary biologic of the animal as the food associated with the non-preferred stimulus least frequently chosen by the animal and/or chosen at a lower response rate and/or chosen after the first stimulus at 706.

One skilled in the art will recognize that each repetition of steps (a) to (d) may be referred to as a trial. It also will be evident to one skilled in the art that the preference test or the association days are not required prior to discrimination learning. Furthermore, it will be apparent to one skilled in the art that if a preference test is not conducted, that random stimulus-food, food stuff or veterinary biologic associations will suffice. Further, if a single stimulus is used, then the response rate to the same stimulus, or to two different stimuli, when associated with different foods, food stuffs or veterinary biologics may be used.

In one embodiment of the invention discrimination conditioning is obtained when the animals passes an a priori two stage-learning criterion. In one embodiment of the invention the two stage-learning criterion comprises: (a) a first stage wherein the animal must choose their preferred stimulus 0-10% of the time in one session, 0-20% of the time over two consecutive sessions, or 0-30% of the time over three sessions; and (b) a second stage wherein the animal must choose their preferred stimulus 0-10% of the time over three sessions subsequent to passing the first stage.

One skilled in the art will recognize that either a more or less stringent criterion, or no criterion, can be used either in a single stage or in a plurality of stages.

In one embodiment of the invention there is a stabilization phase after the discrimination learning procedure. The stabilization phase involves repeating steps (a) to (f) of the discrimination learning procedure. In one embodiment of the invention, the steps are repeated at least 40 times each session for 0-20 sessions, 20-40 sessions or at least 40 sessions. In another one embodiment of the invention, the steps are repeated 20 to 40 times each session for 0-20 sessions, 20-40 sessions or at least 40 sessions. In another embodiment of the invention, the steps are repeated 15-20 times each session for 0-20 sessions, 20-40 sessions or at least 40 sessions. In yet another embodiment of the invention, the steps are repeated 10-15 times each session for 0-20 sessions, 20-40 sessions or at least 40 sessions and in another embodiment the steps are repeated 5-10 or 0-5 times each session for 0-20 sessions, 20-40 sessions or at least 40 sessions.

In yet another embodiment of the invention, the discrimination learning procedure comprises discrimination conditioning comprising: (a) presenting the animal with the preferred stimulus and at least two non-preferred stimuli; (b) permitting the animal to choose one stimulus wherein choice of the preferred stimulus results in no reward and choice of any one non-preferred stimuli result in reward of a particular food, a food stuff, or veterinary biologic; (c) recording the stimulus chosen by the animal and/or the response latency, and/or the order of responses, and/or order of responses, and/or combination thereof; (d) optionally repeating steps (a) through (c) until the results have stabilized; (e) establishing a preferred food, food stuff, or veterinary biologic of the animal as the food, food stuff, or veterinary biologic associated with the non-preferred stimulus chosen most frequently and/or at a higher response rate, and/or chosen first, and/or combination thereof; and, (f) establishing the non-preferred food, foodstuff, or veterinary biologic as the food, food stuff, or veterinary biologic associated with the non-preferred stimulus chosen least frequently, and/or at a lower response rate, and/or after the first stimulus, and/or a combination thereof.

In another embodiment of the invention, the methods described above further steps (a) to (d) are repeated any number of times in one session for as many sessions as is desired until the preferred and non-preferred foods, food stuffs, or veterinary biologics are established as indicated above.

One skilled in the art will recognize that each repetition of steps (a) to (d) may be referred to as a trial. It will also be apparent to one skilled in the art that determination of preference or palatability can be limited to the stabilization phase comprising these steps as shown in FIG. 7, steps 701-704.

In one embodiment of the invention a reversal phase is conducted after the discrimination learning procedure. In yet another embodiment there is a reversal phase conducted after the stabilization phase.

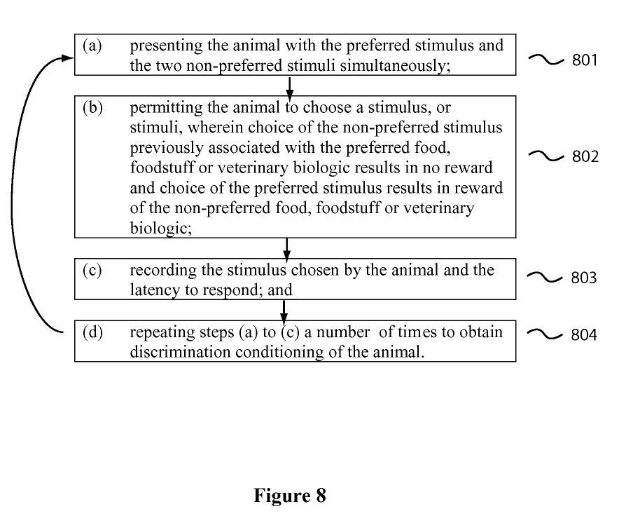

In one embodiment of the invention as shown in FIG. 8 the reversal phase comprises the steps of; (a) presenting the animal with the preferred stimulus and the two non-preferred stimuli simultaneously at 801; (b) permitting the animal to choose a stimulus, or stimuli, wherein choice of the non-preferred stimulus previously associated with the preferred food, foodstuff or veterinary biologic results in no reward and choice of the preferred stimulus results in reward of the non-preferred food, foodstuff or veterinary biologic at 802; (c) recording the stimulus chosen by the animal and the latency to respond at 803; and (d) repeating steps (a) to (c) a plurality of times to obtain discrimination conditioning of the animal at 804.

One skilled in the art will recognize that each repetition of steps (a) to (d) may be referred to as a trial. It will also be apparent to one skilled in the art that any stimulus combination can be used in the reversal phase wherein the associations differ from that of a previous phase. Furthermore, completely different stimuli could be used than in previous phases, and that the reversal phase can occur a plurality of times. A stabilization phase, as described above, can also follow the reversal phase and food preferences may be established in this phase in a similar manner as in the discrimination learning procedure.

One skilled in the art will recognize that a session may be any designated amount of time that is appropriate for the experimental animal being used. For example, one session may comprise at least one trial.

In one embodiment of the invention, the steps are repeated 20 to 10000 times each session for 0-20 sessions, 20-40 sessions or 40-10000 sessions. In another embodiment of the invention, the steps are repeated 15-20 times each session for 0-20 sessions, 20-40 sessions or 40-10000 sessions. In yet another embodiment of the invention, the steps are repeated 10-15 times each session for 0-20 sessions, 20-40 sessions or 40-10000 sessions and in another embodiment the steps are repeated 5-10 or 0-5 times each session for 0-20 sessions, 20-40 sessions or 40-10000 sessions.

In another embodiment of the invention, the methods described above further comprise a reversal phase comprising the steps of: (a) presenting the animal with the preferred stimulus and at least two non-preferred stimulus; (b) permitting the animal to choose one stimulus wherein choice of the non-preferred stimulus previously associated with the preferred food, food stuff or veterinary biologic results in no reward and choice of the preferred stimuli results in reward of the non-preferred food, a food stuff, or veterinary biologic; (c) recording the object chosen by the animal; (d) repeating steps (a) through (c) a plurality of times to obtain discrimination conditioning of the animal.

In yet another embodiment of the invention, the reversal phase comprises the steps of: (a) presenting the animal with the preferred stimulus and at least two non-preferred stimuli simultaneously; (b) permitting the animal to choose one stimulus wherein choice of the preferred stimulus results in no reward and choice of the non-preferred stimuli results in reward of a food, food stuff, or veterinary biologic not previously associated with the non-preferred stimulus; (c) recording the object chosen by the animal, and/or the latency to respond and/or the order of responses; (d) repeating steps (a) through (c) a plurality of times to obtain discrimination conditioning of the animal.

For the reversal phase the establishment of preferred and non-preferred foods may occur as indicated above for the discrimination learning procedure.

The description of the various phases is included for the purpose of teaching those skilled in the art how to practice the invention and is not intended to recite all the possible modifications and variations thereof that will become apparent to the skilled worker upon reading. For example, the number of stimuli may be increased to assess more foods, food stuffs or veterinary biologics concurrently, simultaneously or in parallel. Furthermore, object or other preferences may be assessed using the response frequency, response rate, or order of responses, or a combination thereof. All such modifications and variations are in keeping with the spirit of the invention as claimed herein.

The following test result examples are presented for the purpose of further illustrating and explaining the present invention. These examples are for illustrative purpose and are not intended to be limiting in any regard.

### EXAMPLE 1

First Phase: preference and association testing The single object preference test is used to determine object preferences. Three different objects were presented to the canines for twelve trials, each associated with approximately one gram of a moist food. The positions of the objects were randomized among the three possible well positions ensuring all possible combinations occurred equally within the twelve trials and the number of responses to each object was recorded. The object chosen most often was considered to be the canine's preferred object. In all subsequent testing, the preferred object was associated with non-reward.

Two association sessions followed the preference test. The purpose of the association sessions was to familiarize each canine with the particular objects and the test food associated with it. Each association session was performed on a separate day. On the first association day, each canine received 12 trials in which one of the non-preferred objects was presented over the middle well containing the moist test food. On the second association day, the second non-preferred object was presented for 12 trials covering the middle well, which contained the dry test food. On both association days, only a single object was presented, and displacement of the object was always associated with a particular test food.

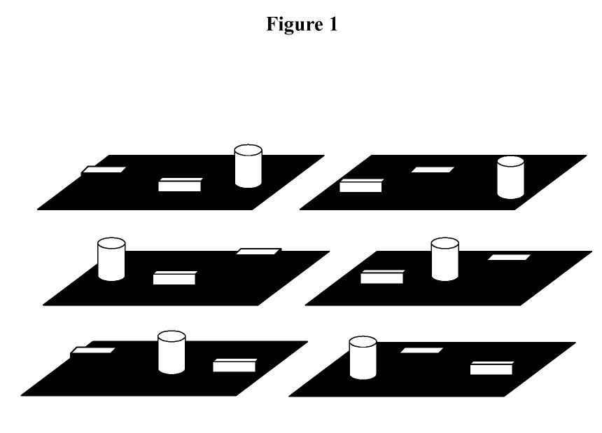

Second Phase: discrimination-learning procedure During the discrimination-learning procedure, the canines were given 12 trials each daily session with an interval of 30 seconds separating each trial. Each trial began with the simultaneous presentation of the three objects to the canine. The location of each object was varied in a quasirandom manner to assure that all possible combinations of object placement occurred equally during a twelve trial test session (FIG. 1). The preferred object, determined in the preference test, was never placed over food, and the remaining objects were always placed over the test food associated with them during the association days. A trial ended after the canine displaced one of the three objects and retrieved the food (unless they responded to the object associated with non-reward).

This phase of the protocol was completed when the canine passed an a priori two stage-learning criterion based on the canine's response to the non-rewarded object. The first stage required the canines to choose their preferred object less than two times during one test session, less than four times over two consecutive test sessions, or less than eight times over three consecutive test sessions. To pass the second stage, the subjects were required to choose their non-preferred object less than ten times over the three consecutive sessions subsequent to passing the first criteria.

Stabilization Phase The stabilization phase was intended to establish the strength and reliability of dietary preferences. Canines were tested for 20 days using a procedure identical to that during the discrimination-learning procedure. The number of choices to each object during this phase was used to establish food preference.

Reversal Phase This test phase was instituted after completing the stabilization phase. The purpose was to determine whether the preferences remained after the objects associated with the two test foods were switched. In this phase, as in the previous phase, the initially preferred object was never associated with any food. The objects associated with the test foods, however, were switched, such that the object associated with the moist test food type in the earlier phases was now associated with the dry test food and vice versa. This phase included association days, a training phase and stability phase, as described above.

Data Analysis The number of choices of the non-preferred objects during the stabilization phases was used to establish the food preference. A food-preference ratio, calculated by dividing the number of choices to the object associated with the moist test food by the sum of the choices of the object associated with the moist and dry test foods, was used to describe the individual data. Using this measure, a score of 0.5 indicated no preference for either food, while a score of 1.0 indicated a complete preference for the moist test food. Statistical analyses were conducted using Statistica 6.0c with significance set to P<0.05. Individual choices over the stabilization days were analyzed using a two-way repeated-measures ANOVA, with test phase (2 factors) and object (3 factors) serving as within-subject variables. Post-hoc analysis was conducted using Tukey's LSD test if appropriate.

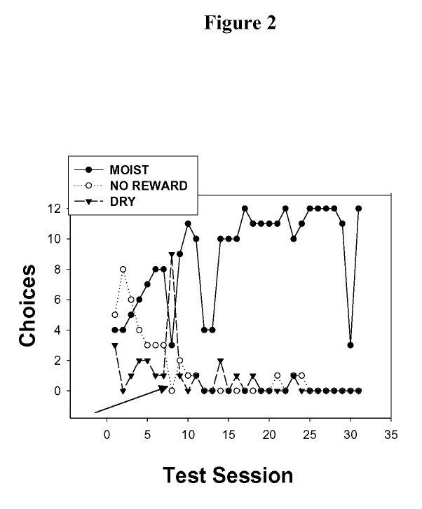

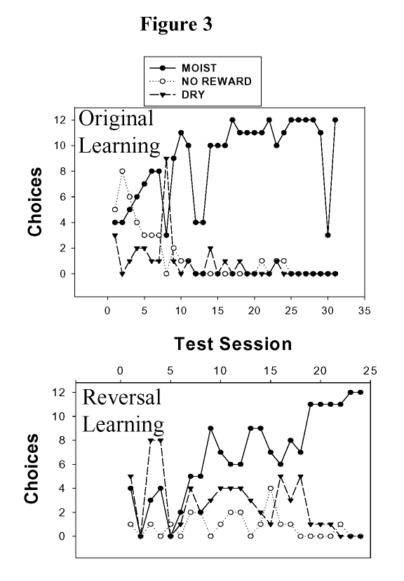

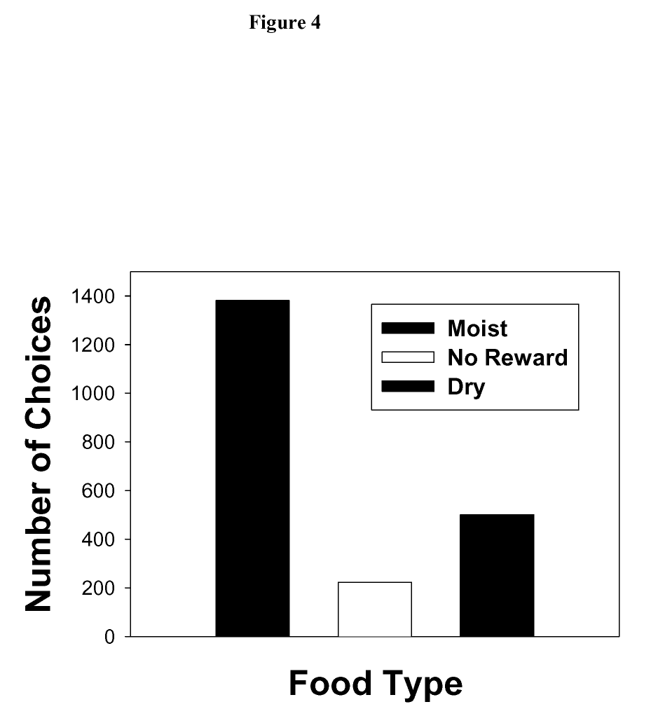

All canines rapidly learned to avoid the object associated with non-reward (FIG. 2). During the subsequent stabilization sessions, every canine showed a very strong and reliable preference for the object associated with the moist food (Table 1). Only four of the five subjects, however, reached criterion after the reversal of objects associated with the two food types. Of these four, three demonstrated an increase in choices of the object associated with the moist food (see FIG. 3). The fourth subject continued to choose the object that was associated previously with the moist test food, despite the changed reward. The overall choices of the moist test food greatly outnumbered the choices of either the dry test food, or the preferred object, over both the initial and reversal phases (FIG. 4). A significant difference between object choices was noted (F2,6=12.88, p<0.001), but not between the initial and reversal test phases. Post-hoc Fisher's indicated that the difference was due to a greater number of choices of the object associated with the moist test food compared to the object associated with the dry test food (p<0.05) and the non-rewarded object (p<0.05). The choices to the object associated with the dry test food and the non-rewarded object did not differ.

Procedure Dogs were trained on a three choice object discrimination learning procedure. After establishing object preferences in the first phase, the preferred object was associated with no reward, a second object was associated with the dry food and the third object was associated with a highly palatable moist food in the second phase. In the second phase, the dogs were trained on the discrimination learning procedure until they learned to avoid the non-rewarded object. They were subsequently given an additional 20 test sessions during a stabilization phase. In the reversal learning phase, the object-food associations were modified, such that the object that was previously associated with moist food was now associated with the dry food, and vice versa. Once the dogs learned to avoid the non-rewarded object, they were tested on the discrimination learning procedure for another 20 sessions during an additional stabilization phase.

Results The object associated with the moist food was chosen to a greater extent than the other objects, indicating a strong preference for the moist food.

Materials and Methods Subjects Two male and three female beagle dogs from our colony at the University of Toronto were used. Two dogs were between three and five years of age and the remaining dogs were between nine and twelve. All the dogs had been in the colony for at least one year and all had previous experience on a variety of tests of cognitive function. The subjects were housed individually in pens measuring approximately 1.07×1.22 m and were fed once daily after palatability testing. Water was available ad libitum. Dogs were maintained on a 12:12-hr light-dark cycle and were exercised daily while their pens were cleaned. All canines underwent regular clinical examinations and had no health problems throughout the duration of the study.

Apparatus A wooden chamber based on the Wisconsin General Test Apparatus (previously described in Milgram et al., 1994) was used for palatability testing. Vertical stainless steel bars, covering the front of the box, provided access to the objects and test foods associated with them. Objects were presented on a sliding Plexiglas tray, which contained one medial and two lateral food wells. The test foods could be accessed by displacement of the appropriate object from above the food well. The dogs and the tester were separated by a wooden screen, which had a hinged door at the bottom, to allow presentation of the sliding tray, and a one-way mirror above, which permitted the tester to view the subject. An incandescent 60-watt light attached to the front of the chamber served as the only source of lighting during testing.

Food Comparisons Two foods were compared in this study: Purina Agribrands Canine Lab Chow #5006a, a dry food, which also served as the regular daily diet for all the subjects, and Hill's Prescription Diet (P/D)_{b}, a moist dog food, intended to be highly palatable.

Design Palatability testing was divided into four phases: a preference and association phase, a discrimination learning phase, a stabilization phase, and a reversal phase.

### EXAMPLE 2

First Phase: preference and association testing The preference test was used to determine object preferences. Three different objects were presented to the canines for twelve trials, each associated with approximately one gram of Hill's P/D diet. The positions of the objects were randomized among the three possible well positions ensuring all possible combinations occurred equally within the twelve trials and the number of responses to each object was recorded. The object chosen most often was considered to be the canine's preferred object. In the subsequent discrimination phase, the preferred object was associated with no reward.

Two association sessions followed the preference test. The purpose of the association sessions was to familiarize each canine with the particular objects and the test food associated with it. On the first association session, each canine received 12 trials in which one of the non-preferred objects was presented over the middle well containing one of the test foods. On the second association session, the second non-preferred object was presented for 12 trials covering the middle well, which contained the other test food. On both association sessions, only a single object was presented, and displacement of the object was always associated with a particular test food.

Second Phase: discrimination-learning procedure During the discrimination-learning procedure, the canines were given 12 trials for each daily session with an interval of 30 seconds separating each trial. Each trial began with the simultaneous presentation of the three objects to the canine. The location of each object was varied in a quasirandom manner to assure that all possible combinations of object placement occurred equally during a twelve trial test session (see FIG. 1 as an example). The preferred object, determined in the preference test, was never placed over food, and the remaining objects were always placed over the test food associated with them during the association days. A trial ended after the canine displaced one of the three objects and retrieved the food (unless they responded to the object associated with no reward).

This phase of the protocol was completed when the canine passed an a priori two stage-learning criterion based on the canine's response to the non-rewarded object. The first stage required the canines to choose their preferred object less than two times during one test session, less than four times over two consecutive test sessions, or less than eight times over three consecutive test sessions. To pass the second stage, the subjects were required to choose their non-preferred object less than ten times over the three consecutive sessions subsequent to passing the first criteria.

Stabilization Phase The stabilization phase was intended to establish the strength and reliability of dietary preferences. Canines were tested for at least 10 days using a procedure identical to that during the discrimination-learning procedure. The number of choices to each object during this phase was used to establish food preference.

Reversal Phase This test phase was instituted after completing the stabilization phase. The purpose was to determine whether the preferences remained after the object food association contingencies were modified. In this phase, the initially preferred object was associated with the non-preferred food, the object associated with the preferred food was not rewarded and the object associated with the non-preferred food was associated with the preferred food. This phase included association days, a training phase and stability phase, as described above.

Data Analysis The number of choices of the non-preferred objects during the stabilization phases was used to establish the food preference. Statistical analyses were conducted using Statistica 6.0c with significance set to P<0.05. In order to analyze food preference, individual one-way repeated-measures ANOVAs were conducted with percentage of choices to each food and non-reward serving as a within-subject variable for the original and reversal learning. Post-hoc analysis was conducted using Tukey's LSD test if appropriate.

All subjects passed the two-phase criterion within 16 test sessions (M=8.3+4.9; Table 2). Table 2 also indicates the number of choices to each food choice. The preference ratio was calculated by dividing the number of choices to chicken-based test food by total number of choices to the chicken- and lamb-based foods. Thus a score of 1 indicates a complete preference for the chicken-based test food and a score of 0 indicates a complete preference for the lamb-based food. A score of 0.5 indicates no preference. As indicated in Table 2, all subjects showed a preference for the chicken-based test food.

When the objects associated with test diets were switched, every animal learned first to avoid the object associated with no reward and subsequently, with repeated testing every animal developed a preference for the object associated with the chicken-based food. Table 3 indicates the number of sessions to pass criterion and the percentage of each food choice and food preferences for the following 10 sessions. As expected, the number of sessions to pass the learning criterion increased. Also, the 2 subjects that had lower preferences ratios developed a stronger preference for the chicken-based test food. The preference of one subject dwindled. The results of the reversal indicate that the subjects are responding based on food preference and not object preference. Furthermore, these results support the hypothesis that food preferences are learned.

For the original learning, a highly significant effect was found (F(2,10)=23.0; p=0.00018). Tukey's analysis indicated that this effect was due to a high percentage of choices to the chicken-based test food compared to the lamb-based test food (p=0.00043) and the non-reward (p=0.00097). For the reversal learning, a large effect was found [F(2,10)=72.0; p=0.000001). Once again, Tukey's analysis indicated this was due to the high percentage of choices to the chicken-based test food compared to the lamb-based test food (p=0.0002) and the non-reward (p=0.0002).

Procedure Dogs were trained on a three choice object discrimination learning procedure. After establishing object preferences in the first phase, the preferred object was associated with non-reward, a second object was associated with the chicken-based test food and the third object was associated with the lamb-based test food in the second phase. Both test foods were similar in appearance and texture and the main difference was the meat flavor. In the second phase, the dogs were trained on the discrimination learning procedure until they learned to avoid the non-rewarded object. They were subsequently given a minimum of 10 additional test sessions, at the very least, during a stabilization phase. In the second phase, which also involved reversal learning, the object-food associations were modified, such that the object that was previously associated with the chicken-based test food was now associated with no reward, the preferred object was associated with the lamb-based test food and the object previously associated with the lamb-based test food was associated with the chicken-based test food. Once the dogs learned to avoid the non-rewarded object, they were tested on the discrimination learning procedure for 10 sessions during an additional stabilization phase.

Results The object associated with the chicken-based test food was chosen to a greater extent than the other objects, indicating a strong preference for this test food.

Materials and Methods Subjects Three male and three female beagle dogs from our colony at the University of Toronto were used. All dogs were less than seven years of age, had been in the colony for at least one year and had previous experience on a variety of tests of cognitive function. The subjects were housed individually in pens measuring approximately 1.07×1.22 m and were fed once daily after palatability testing. Water was available ad libitum. Dogs were maintained on a 12:12-hr light-dark cycle and were exercised daily while their pens were cleaned. All canines underwent regular clinical examinations and had no health problems throughout the duration of the study.

Apparatus A wooden chamber based on the Wisconsin General Test Apparatus (previously described in Milgram et al., 1994) was used for palatability testing. Vertical stainless steel bars, covering the front of the box, provided access to the objects and test foods associated with them. Objects were presented on a sliding Plexiglas tray, which contained one medial and two lateral food wells. The test foods could be accessed by displacement of the appropriate object from above the food well. The dogs and the tester were separated by a wooden screen, which had a hinged door at the bottom, to allow presentation of the sliding tray, and a one-way mirror above, which permitted the tester to view the subject. An incandescent 60-watt light attached to the front of the chamber served as the only source of lighting during testing.

Food Comparisons Two foods were compared in this study: test foods 1 and 2 were both dry kibble, similar in appearance and texture, but were based on lamb and chicken as a meat source, respectively.

Design Palatability testing was divided into four phases: a preference and association phase, a discrimination training phase, a stabilization phase, and a reversal phase.

The above description is for the purpose of teaching those skilled in the art how to practice the invention and is not intended to recite all the possible modifications and variations thereof that will be apparent to one skilled in the art. It is intended, however, that all such modifications and variations be included within the scope of the invention that is defined by the following claims.

Table 1: Results of all subjects on initial learningSubject #12345Sessions to Criterion14101117Responses During Stabilization TrialsMoist P/D215179130201122Non-Reward92651364Dry Chow163158554Preference Ratio2.93.85.69.97.691Number of daily sessions to pass criterion.

2Preference ratios were calculated using the formula:
choices of moist PD/(choices of moist PD+choices of dry chow).

TABLE 2: Overview of Initial LearningSubjectCriterion SessionsPost Criterion YoTo No FoodPost Criterion YoTo Lambbased FoodPost Criterion YoTo Chicken-based FoodPreference RatioMorpheus53.35.890.8.94Apollol203.396.7.97Monk422.830. 946.3.60Celeste42.815.681.7.84Tinkle166.12.291.7.98V aughn94.340.355.6.58Mean 8.36.516.477.1.82TABLE 3: Overview of Reversal LearningSubjectCriterion SessionsPost Criterion YoTo No FoodPost Criterion YoTo Lambbased FoodPost Criterion YoTo Chicken-based FoodPreference RatioMorpheusl31.77.690.8.92Apollo104.21.794.2.98Monk256.7 3.489.9.96Celeste123.4096.61.0Tinkle131.736.761.7.63 Vaughn216.73.390.96Mean 11.674.18.887.2.91

1. A method of determining the palatability of a food, food stuff or veterinary biologic to an animal comprising:
obtaining at least one animal; and,
administering a discrimination learning procedure to an animal selected from said at least one animal wherein said discrimination learning procedure comprises using a food, food stuff or veterinary biologic.

2. The method of claim 1 further comprising utilizing at least one stimulus preference test comprising:
(a) presenting said animal with at least one distinct stimulus wherein each of said at least one distinct stimulus is associated with an identical reward;
(b) permitting said animal to choose from said at least one distinct stimulus, wherein choice of any one stimulus results in said identical reward;
(c) recording a preferred stimulus of said animal wherein said preferred stimulus is most frequently chosen by said animal or chosen at a greater response rate or chosen first; and,
(d) recording a non-preferred stimulus of said animal wherein said non-preferred stimulus is least frequently chosen by said animal or chosen at a slower response rate or chosen secondly or later.

3. The method of claim 1 further comprising utilizing at least one association session comprising:
(a) presenting said animal with at least one stimulus associated with said food, food stuff or veterinary biologic; and,
(b) permitting said animal to choose from said at least one stimulus wherein any choice results in a reward of said food, food stuff or veterinary biologic associated with said any choice.

4. The method of claim 1 wherein said administering said discrimination learning procedure comprises:
(a) presenting said animal with a preferred stimulus and a plurality of non-preferred stimuli;
(b) permitting said animal to choose at least one stimulus selected from said preferred stimulus and said plurality of non-preferred stimuli wherein choice of any one of said non-preferred stimuli results in a reward of said food, food stuff or veterinary biologic associated with a corresponding said any one of said non-preferred stimulus and choice of a preferred stimulus results in no reward;
(c) recording said at least one stimulus chosen by said animal; and,
(d) repeating steps (a) through (c) to obtain discrimination conditioning of said animal.

5. The method of claim 4 further comprising establishing a preferred food, foodstuff, or veterinary biologic of said animal as said food, foodstuff, or veterinary biologic associated with a stimulus most frequently chosen or chosen most rapidly or chosen first or an association learned most rapidly by said animal.

6. The method of claim 5 further comprising establishing a rank order of preferred food, foodstuff, or veterinary biologic of said animal as an ordered list of decreasing preference of said food, foodstuff, or veterinary biologic associated with a stimulus most frequently chosen or chosen most rapidly or chosen first or an association learned most rapidly by said animal.

7. The method of claim 4 further comprising establishing a non-preferred food, foodstuff, or veterinary biologic of said animal as said food, food stuff or veterinary biologic associated with a stimulus least frequently chosen or chosen more slowly or chosen following a stimulus associated with said preferred food, food stuff or veterinary biologic or an association learned less rapidly.

8. The method of claim 7 further comprising establishing a rank order of non-preferred food, foodstuff, or veterinary biologic of said animal as an ordered list of increasing preference of said food, food stuff or veterinary biologic associated with a stimulus least frequently chosen or chosen more slowly or chosen following a stimulus associated with said preferred food, food stuff or veterinary biologic or an association learned less rapidly.

9. The method of claim 4 further comprising utilizing a stabilization phase wherein steps (a) to (d) are repeated at least once in one session for at least one consecutive sessions.

10. The method of claim 1 further comprising utilizing a reversal phase comprising:
(a) presenting said animal with a preferred stimulus and a plurality of non-preferred stimuli;
(b) permitting said animal to choose a stimulus wherein choice of a non-preferred stimulus previously associated with said preferred food, food stuff or veterinary biologic results in no reward and choice of said preferred stimulus results in a reward of said food, a food stuff, or veterinary biologic not previously associated with said preferred stimulus;
(c) recording a selected stimulus chosen by said animal or a latency to respond or an order of responses;
(d) repeating steps (a) through (c) to obtain discrimination conditioning of said animal.

11. The method of claim 10 further comprising establishing a preferred food, foodstuff, or veterinary biologic of said animal as said food, foodstuff, or veterinary biologic associated with a stimulus most frequently chosen by said animal or chosen most rapidly or chosen first or an association learned most rapidly.

12. The method of claim 10 further comprising establishing a non-preferred food, foodstuff, or veterinary biologic of said animal as said food, food stuff or veterinary biologic associated with a stimulus least frequently chosen or chosen more slowly or chosen following a stimulus associated with said preferred food, food stuff or veterinary biologic or an association learned less rapidly.

13. A system for determining the palatability of a food, food stuff or veterinary biologic to an animal comprising:
means for obtaining at least one animal; and,
means for administering a discrimination learning procedure to an animal selected from said at least one animal wherein said discrimination learning procedure comprises means for using a food, food stuff or veterinary biologic.

14. The system of claim 13 further comprising means for utilizing at least one stimulus preference test comprising:
(a) means for presenting said animal with at least one distinct stimulus wherein each of said at least one distinct stimulus is associated with an identical reward;
(b) means for permitting said animal to choose from said at least one distinct stimulus, wherein choice of any one stimulus results in said identical reward;
(c) means for recording a preferred stimulus of said animal wherein said preferred stimulus is most frequently chosen by said animal or chosen at a greater response rate or chosen first; and,
(d) means for recording a non-preferred stimulus of said animal wherein said non-preferred stimulus is least frequently chosen by said animal or chosen at a slower response rate or chosen secondly or later.

15. The system of claim 13 further comprising means for utilizing at least one association session comprising:
(a) means for presenting said animal with at least one stimulus associated with said food, food stuff or veterinary biologic; and,
(b) means for permitting said animal to choose from said at least one stimulus wherein any choice results in a reward of said food, food stuff or veterinary biologic associated with said any choice.

16. The system of claim 13 wherein said means for administering said discrimination learning procedure comprises:
(a) means for presenting said animal with a preferred stimulus and a plurality of non-preferred stimuli;
(b) means for permitting said animal to choose at least one stimulus selected from said preferred stimulus and said plurality of non-preferred stimuli wherein choice of any one of said non-preferred stimuli results in a reward of said food, food stuff or veterinary biologic associated with a corresponding said any one of said non-preferred stimulus and choice of a preferred stimulus results in no reward;
(c) means for recording said at least one stimulus chosen by said animal.

17. The system of claim 16 further comprising means for establishing a preferred food, foodstuff, or veterinary biologic of said animal as said food, foodstuff, or veterinary biologic associated with a stimulus most frequently chosen or chosen most rapidly or chosen first or an association learned most rapidly by said animal.

18. The system of claim 17 further comprising means for establishing a rank order of preferred food, foodstuff, or veterinary biologic of said animal as an ordered list of decreasing preference of said food, foodstuff, or veterinary biologic associated with a stimulus most frequently chosen or chosen most rapidly or chosen first or an association learned most rapidly by said animal.

19. The system of claim 16 further comprising means for establishing a non-preferred food, foodstuff, or veterinary biologic of said animal as said food, food stuff or veterinary biologic associated with a stimulus least frequently chosen or chosen more slowly or chosen following a stimulus associated with said preferred food, food stuff or veterinary biologic or an association learned less rapidly.

20. The system of claim 19 further comprising means for establishing a rank order of non-preferred food, foodstuff, or veterinary biologic of said animal as an ordered list of increasing preference of said food, food stuff or veterinary biologic associated with a stimulus least frequently chosen or chosen more slowly or chosen following a stimulus associated with said preferred food, food stuff or veterinary biologic or an association learned less rapidly.

21. The system of claim 16 further comprising means for utilizing a stabilization phase.

22. The system of claim 13 further comprising means for utilizing a reversal phase comprising:
(a) means for presenting said animal with a preferred stimulus and a plurality of non-preferred stimuli;
(b) means for permitting said animal to choose a stimulus wherein choice of a non-preferred stimulus previously associated with said preferred food, food stuff or veterinary biologic results in no reward and choice of said preferred stimulus results in a reward of said food, a food stuff, or veterinary biologic not previously associated with said preferred stimulus;
(c) means for recording a selected stimulus chosen by said animal or a latency to respond or an order of responses.

23. The system of claim 22 further comprising means for establishing a preferred food, foodstuff, or veterinary biologic of said animal as said food, foodstuff, or veterinary biologic associated with a stimulus most frequently chosen by said animal or chosen most rapidly or chosen first or an association learned most rapidly.

24. The system of claim 22 further comprising means for establishing a non-preferred food, foodstuff, or veterinary biologic of said animal as said food, food stuff or veterinary biologic associated with a stimulus least frequently chosen or chosen more slowly or chosen following a stimulus associated with said preferred food, food stuff or veterinary biologic or an association learned less rapidly.

25. A product created by process of determining the palatability of a food, food stuff or veterinary biologic to an animal comprising:
a composition comprising an enhanced palatability or more preferable to an animal wherein said composition is selected in accordance with the method of claim 1.

26. A product created by process of determining the palatability of a food, food stuff or veterinary biologic to an animal comprising:
a composition comprising a reduced palatability or less preferable to an animal wherein said composition is selected in accordance with the method of claim 1.
